(19) United States
(12) Patent Application Publication
(10) Pub. No.: US 2024/0331841 A1
(43) Pub. Date: Oct. 3, 2024

(54) VIRTUAL REALITY ENABLED NEUROTHERAPY FOR IMPROVING SPATIAL-TEMPORAL NEUROCOGNITIVE PROCESSING

(71) Applicant: REACT Neuro Inc., Cambridge, MA (US)

(72) Inventors: Anthony J. Simon, Davis, CA (US); Theodore M. Aronson, Davis, CA (US)

(73) Assignee: REACT Neuro Inc., Cambridge, MA (US)

(21) Appl. No.: 18/499,979

(22) Filed: Nov. 1, 2023

Related U.S. Application Data
(63) Continuation of application No. 17/261,543, filed on Jan. 19, 2021, now abandoned.
(60) Provisional application No. 62/714,428, filed on Aug. 3, 2018.

(51) Int. Cl. G16H 20/70; A63F 13/53; A63F 13/57; A63F 13/67; A63F 13/80

(52) U.S. Cl. CPC G16H 20/70; A63F 13/53; A63F 13/57; A63F 13/67; A63F 13/80; A63F 2300/8082

(57) ABSTRACT

Provided herein are methods, tools, devices and systems for treating, enhancing and improving spatial and temporal neurocognitive processing abilities and associated neuromotor activities of a subject, including a subject suffering from a neurocognitive disorder or condition such as a stroke, brain injury or genetic disorder.

### CROSS-REFERENCE TO RELATED APPLICATIONS

This application claims priority from U.S. provisional application No. 62/714,428, filed Aug. 3, 2018, entitled “VIRTUAL REALITY ENABLED NEUROTHERAPY FOR IMPROVING SPATIAL-TEMPORAL NEUROCOGNITIVE PROCESSING,” the contents of which are incorporated by reference in their entirety.

### BACKGROUND

Dysfunctions in spatial and temporal information processing contribute to debilitating functional impairments in a number of diseases, conditions and circumstances. Such dysfunctions result from many types of stroke and brain injury where they induce significant, and functionally limiting, impairments in cognitive and motor functions that require accurate spatial and temporal mental representations of the information being processed. They can be caused by atypical brain development in one of several clearly specified genetic disorders including chromosome 22q11.2 Deletion, Turner, fragile X and Williams syndromes. The result is learning difficulties, especially in the domain of quantitative and numerical thinking, as well as functions like reading and conditions like dyslexia. Additionally, degrading brain structure and function that occurs in aging humans, and in some neurodegenerative disorders, results in reduced spatiotemporal abilities that also have effects on cognitive functions. These include memory, attention, and neuromotor functions such as gait variability that contributes to falls and other accident risk. There is a need for therapeutic methods that can address spatial and temporal impairments and that can produce long-lasting improvement in spatial and temporal neurocognitive processes and associated neuromotor activities.

### SUMMARY

Provided herein are methods, tools, devices and systems for treating, enhancing and improving spatial and temporal neurocognitive processing abilities and associated neurornotor activities of a subject using virtual reality-based therapy.

### BRIEF DESCRIPTION OF THE DRAWINGS

FIG. 1 presents an illustration of a generation algorithm.

FIG. 2 presents a flow chart illustrating how the systems described herein performs the adaptation process in response to a subject's response.

FIG. 3 illustrates the compilation from an exemplary subject of a range of motion values measured by an embodiment of the system described herein.

FIG. 4 illustrates a measurement of an exemplary subject's spectral arc length for a measured treatment activity using an embodiment of the system described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

Provided herein are methods, tools and systems for neurocognitive therapy. The therapy employs virtual reality methods to target spatial and temporal neurocognitive systems and thereby improve their functioning in a subject. The treatment is delivered through a virtual reality hardware platform that provides advantage over a two-dimensional display (such as a computer, mobile computing device, or television console) which have fixed screens with a limited field of view and which require unnatural physical responses of a subject (such as touching a screen or pressing buttons or manipulating a joystick on a game controller that differ from everyday neuromotor responses). The virtual reality therapy (VR therapy) provided herein has a 3D view and is responsive to a subject's neuromotor interaction with the system. Fully immersive audio can also be included to relay spatial and temporal information. VR therapy activates a subject's target neural and motor systems in a naturalistic, integrated fashion, thereby creating a seamless link between activities carried out during treatment and as a result, the implementation of these same functions in the subject for a wide range of real world behaviors.

A virtual reality (VR) system such as provided herein has advantages as compared to 2D systems that make it a valuable platform for the delivery of therapeutic stimulation to the central nervous system of a subject, most especially when a subject's central nervous system is sufficiently damaged to preclude effective neurocognitive and neuromotor functioning in the real world. A VR system presents information to at least three senses (sight, sound and touch) to the brain in a fully immersive, 3D manner that directly parallels the real world. Therefore, the brain responds in the same way to that information in VR as it does in reality. Neuromotor responses to information presented in VR are close parallels to, if not exact matches of, the actions taken in reality. Visual information within the headset can be scanned with covert (without eye movement) and overt (with eye movement) attentional actions as in the real world. VR provides a larger field of view as compared to 2D systems. With VR, visual and auditory information are both available in full 360 degree space and are detected, localized, attended to and examined in the same way as in the real world, with head and body movements and with timing of auditory input to each ear. Looking away from the center of a 2D display causes it to disappear from view and cuts off all visual and much auditory information flow to the brain. With VR, a range of input devices enable the user to respond to objects and events in VR in completely naturalistic ways through movements of the head, trunk, arms, hands, fingers and even legs, feet and toes in future instantiations. Communication between the brain and body throughout the full “perception action cycle” is naturalistic and embodied, meaning that inputs and outputs are processed by a subject just as they are in the real world.

VR allows for a subject's total control of the physics of the space and the objects events in it, which also cannot be done in 2D environments in such a complete manner. A subject's actions and movements in VR can be amplified or reduced in order to let the subject experience things or execute actions they are unable to do in the real world. For example, a stroke patient who can barely move a hemiplegic arm can experience moving the virtual instantiation of that arm a lot, much faster, more accurately, more stably etc. In another example, a subject's inability to control her hand and fingers in order to pick up and grasp an object can be given that ability in VR just by moving the virtual hand to within a given proximity to a target item for a given duration, both of which values can be adaptively adjusted as the patient's abilities improve.

The systems and methods provided herein utilize the advantages of VR presentation to provide VR therapy. Provided herein are embodiments of VR therapy that target spatial and temporal neurocognitive systems and improve their functioning. In some embodiments, the therapy reduces one or more functional impairments of a subject, such as one or more spatial and/or temporal neurocognitive impairments. In some embodiments, the VR therapy reduces functional impairments of a subject who has suffered one or more strokes. In some embodiments, the VR therapy reduces functional impairments of a subject who has a brain injury. In other embodiments, the VR therapy provided herein reduces one or more functional disabilities such as learning disabilities resulting from one of several neurodevelopmental disorders that have clear genetic etiology. In other embodiments the VR therapy provided herein reduces functional impairment of an aging subject such as a high fall-risk senior citizen. In some embodiments, the VR therapy herein provides treatment for movement and neurodegenerative disorders such as Parkinson's, Huntington's disease and Multiple Sclerosis.

Provided herein are methods, tools, devices and systems of VR therapy that deliver therapeutic stimulation, which is constantly and dynamically adapted to the subject's abilities in that exact moment. The VR therapy provided herein affects and changes how the human brain processes information about space and time (i.e. spatiotemporal information). Such constantly-adjusted stimulation is personalized (digital) medicine in the purest sense of the term. The VR therapy provided herein achieves spatial and temporal neurocognitive improvements in a subject that cannot be achieved with entertainment-type video games, which primarily seek to extend playing time without intentionally seeking to alter subject capabilities. The VR therapy provided herein also achieves spatial and temporal neurocognitive improvements that differ from “brain fitness” or “brain enhancement” games, which primarily serve as measurement tools or that generate better subject performance in isolated activities largely through practice effects that do not transfer to untrained activities. Because the treatments in the current invention are designed to improve the functioning and capacity of the actual neurocognitive underpinnings of specified mental activities, the VR therapy provided herein achieves long-lasting effects on a subject that translate beyond the subject's interaction with the virtual reality “game” and into real-world activities for improvements in daily life activities.

The adaptive fashion of the methods, tools and systems herein provides for treatment titration for a subject in a manner that exceeds what can be provided through traditional therapy by a highly-trained clinician. The methods, tools and systems herein provide finely tuned measurements of progress that exceed parallel human-based analyses. The methods, tools and systems herein provide continuous testing to determine multiple aspects of the current functional capabilities of the subject being treated beyond traditional therapy provided by a highly-trained clinician.

Provided herein are VR therapy tools, methods and systems that contact and stimulate specific neurocognitive systems to create cognitive and behavioral outcomes. In some embodiments, the outcomes include an improved spatial and/or temporal resolution. As used herein, improved spatial and/or temporal resolution refers to amount of detailed information about real world space and its contents that a subject can mentally represent and then process in the service of achieving a range of behavioral goals. In some embodiments, the outcomes include an improved resolution or amount of detailed information about real world time and its sub-units that a subject can mentally represent and then process in the service of achieving a range of behavioral goals. In some embodiments, the outcomes include an expansion of the proportion of the visual field from within which a subject can detect objects and events in order to mentally represent them at high resolution so that those representations can subsequently be processed in the service of achieving a range of behavioral goals. In some embodiments, the outcomes include an improved accuracy of controlled neuromotor actions based on improved spatial and temporal computations carried out by the brain and associated motor systems. In some embodiments, the VR therapy achieves at least one of these outcomes. In some embodiments the therapies provided herein achieve a combination two or more of these outcomes.

Provided herein are VR therapy methods, tools and systems that address, enhance and/or improve a subject's crowding threshold. When multiple objects or events in space and/or time are mentally represented at a coarse enough resolution (i.e. where some real-world information is lost or degraded in the process of creating internal mental representations from incoming sensory information) that each of those objects or events cannot be stored as uniquely separable representational units, the phenomenon of “crowding” is said to have occurred. The precise specification of the spatial and temporal information required by a specific individual to be able to individuate the items or events, which can be objectively quantified, and/or processed in a deeper fashion as distinct, separable items or events can be defined as that subject's current crowding threshold.

In the VR therapy provided herein initial crowding thresholds for each subject will be determined by initiating play at a level at which most subjects in a given indicated population will be able to quickly engage with and succeed at the simplest challenges presented. In some embodiments, the initial challenge levels are first determined via informal pilot testing with representative sampling from the given population. In the VR therapy provided herein, the optimal level of challenge for a given subject during a specific play session is determined by analyzing a subject's responses and comparing them to performance criteria. The treatment algorithms then continually and adaptively optimize stimulation in a dynamic fashion by adjusting difficulty and challenge of critical spatiotemporal tasks to the current abilities of the given subject.

In the VR therapy provided herein, analyses of a subject's responses and resulting adaptations in challenge, and therefore stimulation, are made on the basis of currently demonstrated spatial and/or temporal cognitive capabilities of the subject during the current treatment (i.e. play) session. In some embodiments of the VR therapy provided herein analyses of a subject's responses and resulting adaptations in challenge are made solely on the basis of measurements made during an on-going play session. At the end of each session, subject characteristics are saved. In some embodiments, the VR therapy includes one or more subsequent sessions of play which start at a challenge level optimized to the saved characteristics from the prevision session.

Provided herein are VR therapy methods, tools and systems that address, enhance and/or improve a subject's spatial crowding threshold. A spatial crowding threshold is determined by the amount of space, measured in degrees of visual angle (DVA) between two (or more) objects or events that determines whether or not one or more of the objects or events can be mentally represented as distinct and separate units, and thus subsequently taken as distinct inputs to other cognitive processes. Above that threshold, a subject will be able to attend to and process information about an object or event appearing at location A and still be aware of another object (or objects) or event (or events) appearing at a distinct location B (and, where applicable, C, D . . . ) because the object(s) or event(s) will be visible at a spatial distance large enough for those objects or event(s) to be perceived and mentally represented as distinct entities. Below that threshold (i.e. at a measurably smaller distance from location A) any other objects or events that appear will be visible but will not be perceived or processed as distinct entities by the subject's cognitive machinery.

A temporal crowding threshold is determined by the amount of time (i.e. duration), measured in milliseconds, of the interval between the appearance of two (or more) objects or events that determines whether or not one or more of the objects or events can be mentally represented as distinct and separate units, and thus subsequently taken as distinct inputs to other cognitive processes. Above that threshold, a subject is able to attend to and process information about and object or event appearing at timepoint A and still be aware of another object(s) or event(s) appearing at the later timepoint B (and, where applicable, C, D . . . ) because the new object(s) or event(s) will be appear following a time duration long enough after timepoint A to be perceived and mentally represented as distinct entities. Below that threshold (i.e. following a measurably shorter duration from timepoint A) any other objects or events that appear will be visible but will not be perceived or processed as distinct entities by a subject's cognitive machinery.

A useful field of view (UFOV) threshold is a specific measurement of the distribution in space, viewed at a specified distance, over which an individual's attention can be spread. Practically this means it is a specification of the limit, in spatial terms, of how far spread apart two or more objects can be while still being viewed at the same time. This threshold interacts significantly with spatial crowding because the resolution of information mentally represented from sensory inputs gathered from the UFOV drops off dramatically at even short distances from the center of the UFOV, which is the point at which inputs from both of the viewer's eyes converge and which generally covers 2 degrees of visual angle. A small UFOV will provide for only a very small area of the viewer's visual field from within which information can be represented at high resolution. Above that threshold, a subject will be able to focus on point A and still be aware of another object (or objects) or event (or events) at point B (and, where applicable, C, D . . . ) because the new object(s) or event(s) will be close enough to point A to be perceived. Below that threshold (i.e. at a greater spatial distance from point A) any other objects or events that appear will be visible but will not be perceived or processed by the subject's cognitive machinery.

Imprecision refers to the circumstance when multiple objects or events in space and/or time are mentally represented at a coarse enough resolution, (i.e. where some real-world information is lost or degraded in the process of creating internal mental representations from incoming sensory information) that the spatial distance or temporal duration between each of those objects or events cannot be accurately computed. The measurements of imprecision and crowding are related: Crowding is a measure of the spatial and temporal relationships between the objects or events; imprecision is a measure of a subject's abilities and/or behavioral responses to the spatial and temporal distribution of the objects or events.

An individual's imprecision threshold refers to the precise specification of the spatial and temporal information required by the individual to accurately represent the spatial or temporal distance between separate items. As an individual's level of imprecision increases, it approaches an imprecision threshold, above which the individual is considered impaired. An imprecision threshold can be determined and objectively quantified.

A spatial imprecision threshold is determined by the difference between the actual amount of space, measured in degrees of visual angle (DVA) or in millimeters, between two (or more) objects and the mentally represented amount of space that is sufficient to determine the accuracy of an action (such as grasping) targeted at one of those objects. Such action will be executed by neuromotor processes taking the mental representation of the spatial information as an input to computations that require sufficient accuracy to execute the intended action. Below that threshold, a subject will be able to accurately represent and process information about the spatial distance between object or event appearing at location A and another object(s) or event(s) appearing at a distinct location B (and, where applicable, C, D . . . ) because the error term in the spatial distance between the object(s) or event(s) will be small enough for the action executed based on that representation by other neuromotor process to be performed accurately. One example is the distance between a cup and an individual's hand that is represented with a small enough error term for the processes necessary for the action of picking up the cup to be computed accurately such that the cup can be picked up. Above that threshold, a subject will not be able to accurately represent and process information about the spatial distance between object or event appearing at location A and one or more object(s) or event(s) appearing at one or more distinct location(s) because the error term in the spatial distance between the object(s) or event(s) will be large enough such that the action executed based on that representation by other neuromotor process cannot be performed accurately. For example, the distance between a cup and an individual's hand is represented with a large enough error term that the processes necessary for the action of picking up the cup cannot be computed accurately such that the individual can pick up the cup.

A temporal imprecision threshold is determined by the difference between the actual amount of time, measured in milliseconds between the occurrence of two (or more) events and the mentally represented amount of time that is sufficient to determine the accuracy of an action (such as catching) targeted at one of those objects. That action will be executed by other neuromotor processes taking the mental representation of the temporal information as an input to computations that require sufficient accuracy to execute the intended action. Below that threshold, a subject will be able to accurately represent and process information about the temporal duration between object or event occurring at Time A and another event (or events) occurring at a distinct Time B (and, where applicable, C, D . . . ) because the error term in the duration between the event(s) will be small enough for the action executed based on that representation by other neuromotor process to be performed accurately. For example, the time between dropping a ball from one hand and it reaching the other hand that is represented with a small enough error term for the processes necessary for the action of catching the ball to be computed accurately such that the ball can be caught with the other hand. Above that threshold, the individual will not able to accurately represent and process information about the temporal duration between object or event occurring at Time A and another event (or events) occurring at a distinct Time B (and, where applicable, C, D . . . ) because the error term in the duration between the event(s) will be large enough that the action executed based on that representation by other neuromotor process cannot be performed accurately. For example, the time between dropping a ball from one hand and it reaching the other hand is represented with a large enough error term that the processes necessary for the action of catching the ball cannot be computed accurately such that the individual can catch the ball with the other hand.

Described herein are methods, tools and systems that provide or participate in a method whereby the neurotherapeutic effect of the treatment are maximized by using Virtual Reality (VR) computing systems to deliver dynamically optimized stimulation to the target neural systems. The application of VR computing systems, tools and methods overcomes limitations of systems which employ flat-screen or 2-dimensional representations and often place the individual in an unnatural, usually seated position, with computing systems for motor and cognitive responses which often require more dynamic and 3-dimensional interactions. The application of VR computing systems, tools and methods also overcome the limitation of employing flat-screen or 2-dimensional representations that requires the subject to undertake actions (such as touching a screen or pressing buttons or manipulating a joystick on a game controller) that differ from everyday neuromotor responses.

The methods, tools and systems provided herein employ “embodied cognition.” Cognition is embodied when it is deeply dependent upon features of the physical body of an individual, that is, when aspects of the individual's body beyond the brain play a significant causal or physically constitutive role in cognitive processing. A majority of forms of adaptive behavior require the processing of streams of sensory information and their transduction into a series of goal-directed actions in which the processing of sensory-guided sequential actions flows from sensory to motor structures, with feedback at every level. At cortical levels, information flows in circular fashion to constitute the “perception—action cycle.” Embodied cognition provides real-world experiences in the sense that, just as in the real world, it does not treat the brain as operating independently. Instead, embodied cognition addresses the interaction of the brain and the other parts of the body with which the brain naturally interacts. For example in the VR systems and methods herein, the subject can respond to prompts from the system to move a hand, arm, head, trunk or other body part to carry out a task (such as catching a ball). These motions and the cognition involved in making such motions mimics motions, and the brain's control of them, the subject would make as if an actual ball had been tossed in the real world. In this manner, the brain must not only move a limb but must have an understanding of where in space (or in time), a limb or appendage sits relative to the task e.g., relative to the space and temporal positions of the ball). Such embodied cognition can be utilized for treatment of conditions where a subject has lost or suffered a reduction in the perception of space or time as it relates to limb motion and positioning, such as with a subject who has suffered a stroke or other brain traumatic injury.

As described herein, the methods, tools and systems provided use VR to engage all systems involved in the perception-action cycle in a manner that is not possible when using fixed and two-dimensional systems such as a computer or mobile device (e.g., smart phone or tablet) as the display and responses systems. The methods, tools and systems provide a significantly more enriched and naturalistic spatial and temporal information. The methods, tools and systems enable a much wider range of mental and physical responses in sitting and/or standing positions that include head, arm, trunk and whole-body movements. In some embodiments, actions enabled include pointing, reaching, catching, throwing and punching. In some embodiments herein include coordination of one or both arms. In some embodiments, the actions include rapid changes in body position(s), for example, carrying out actions represented in the visual world like shooting a bow and arrow, hitting a ball with a baseball bat, rowing a boat, paddling a kayak, steering a car, swimming, pumping one's arms as if running, casting a fishing rod or catching an insect in a net.

The VR systems, tools and methods herein allow precise control of the physics of the virtual world, in a manner that is not achieved by real-world every day circumstances, so that the amount of movement, its precision and other involved factors can be controlled in part or entirely controlled and adapted in response to the subject's individual abilities. For example, the VR systems, tools and methods herein present challenges that can be adapted to the subject as the subject improves in neuromotor responses throughout the therapy for actions such as grabbing an object which is far away from the subject's hand, shooting at and hitting a target for which the subject's aim is current poor/inaccurate, and moving an object out of the way to avoid colliding with another.

Provided herein are methods, tools and systems that through the use of VR encompass embodied cognition and provide a therapeutic effect. In some embodiments, the therapeutic effect is provided by one or more of visual, auditory, motor and emotional experiences. In some embodiments, the VR employed is similar to what is experienced in the real world by an individual such as by using 360-degree presentation of information in a manner that the human brain responds in a similar manner to related experiences in the natural worlds. In some embodiments, the methods, tools and systems provide for a pattern of brain responses of the subject that does not occur when similar activities are presented on a 2-dimensional screen.

In some embodiments, the VR employed herein with the methods, tools and systems, activates the autonomic nervous system, which functions below the level of consciousness to regulate bodily, survival and emotional functions as well as the central and peripheral nervous systems.

In some embodiments herein, the methods, tools and systems address one or more impairments of a subject. In some embodiments, the impairment is a natural degradation of spatial and temporal processing abilities in aging adults. In some embodiments, the impairment is significant, and sometimes extreme, reduction in those abilities that result from acquired brain injury such as in many cases of stroke or traumatic brain injury (TBI). In some embodiments, the impairment is an impairment in spatial and/or temporal information processing that contributes to learning difficulties and developmental delay in children. In some embodiments, the impairment is a neurodevelopmental disorder.

In some embodiments, the impairment of a subject may arise from reductions in the resolution of mental representations for spatial and temporal information in the minds and brains of an affected individual. In some embodiments, the impairment compromises the functioning in an affected individual of domains of higher cognitive function that depend on lower level functions. In some embodiments, the impairment cognitive impairments can be linked to specific anomalies in developing brain structure, or damage to or degradation of fully functional brain structures, such as those implicated in the role of neural circuitry crucial to the representation and processing of spatial and temporal information.

The methods, tools and systems provided herein use an immersive and motivating environment of key characteristics of action and related adaptively controlled situations, delivered in virtual reality format, to generate mental activity in the subject specifically targeted at enhancing spatiotemporal neurocognitive functioning. In some embodiments, the controlled situations are presented in a game-like format. The methods, tools and systems provided herein construct a specific “active compound” (the precise characteristics of the therapeutic VR neurocognitive requirements) targeted to specified neurocognitive functions and mental representations, which constitute the necessary “receptors” (the precise neurocognitive systems in which impairments occur) through a clearly defined delivery vehicle through the mechanics and interactive experience of key characteristics of an action situation and related adaptively controlled situations presented in a VR format. In some embodiments, the situations are presented in an action game-like format (AGF) and may include game-like responses of the subject.

In some embodiments, one component includes specialized adaptation of the key characteristics of AGF and related adaptively controlled game responses to deliver targeted neurotherapeutic stimulation to a subject. In embodiments herein, interaction will always be in first-person point of view (FP-POV) mode such that the subject perceives what is seen and heard as actual environment inputs that respond to a subject's movements. In some embodiments, the VR environment created responds to head movements of the subject, such as a movement of the subject's head to look around. In some embodiments, the VR employed provides for 360-degree spatial sound inputs consistent with the movement of the head. In some embodiments, the VR environment created includes virtual representations of the subject's arms and hands (and sometimes legs and feet) that can be seen by the subject so that there is perfect coherence between the subject's physical movements and those of the virtual body parts that the subject controls through the systems, tools and methods herein. In such an embodiment, the position and orientation of the subject's arms, legs, and feet may be estimated using the position and orientation of the subject's hands and head as well as knowledge of the subject's physical dimensions (e.g. arm length, torso length, shoulder span, etc). Alternatively, the subject may be fitted with infrared, ultrasonic, or other tracking devices which provide information about the position and orientation of the subject's limbs.

In some embodiments herein, initial parameters are set and then a log of all of the subject's actions is created and continually analyzed to determine patterns that relate solely to whether specific “hit” or “miss” criteria are being met by the subject. These are determined by the level of the “game” being played at the time and the characteristics of the subject. The technique generally operates as follows. Initially, the treatment game presents all challenges with difficulty parameters accessible to most subjects as described above and defined by the research studies. Only specific algorithmic values of game play are changed based on continuous dynamic elements of the subject's performance and the initial crowding and imprecision thresholds.

In some embodiments herein, to determine which values are changed (and, more broadly, to determine the challenges presented to a subject), the methods, tools and systems herein determine a set of “cognitive abilities” that relate to the larger concept of spatiotemporal cognition. These abilities are highly specific, interrelated, and each covers a certain aspect of properly perceiving, mentally representing and computing information about space and time. Some examples of these abilities include for example, the ability to resolve detail about an object or event in the presence other objects or events that occur closely in space or time, the ability to distinguish detail of a certain visual size, and the ability to execute a motor activity with spatial and temporal accuracy. Some examples of related abilities include the ability to identify the existence of objects or events in an individual's periphery and the ability to distinguish detail about an object or event in a subject's periphery, and include abilities that rely on vision at the edge of a subject's useful field of view (UFOV).

In some embodiments, the therapeutic games provided herein challenge one or more of these abilities in a subject. At the time that the challenge is generated (e.g. a target appears on screen for the subject to shoot), the capacity levels required to succeed at the challenge are determined. These capacity levels are quantitative and related to physical aspects of the challenge. For example, in a therapeutic game that includes shooting a target that briefly appears and disappears, the capacity level for the ability to identify the existence of an object or event in the periphery is directly related to the spatial eccentricity with respect to the center of the viewing field of the location of the target, and the capacity level for the ability to respond to specific temporal durations is related to the length of time that the target appears on the screen or in auditory space. When the subject succeeds or fails at a challenge, that success or failure is logged within the subject's profile along with the capacity levels that challenge required.

In an exemplary embodiment of a subject catching a ball that is dropped by a device or the subject's other hand, the capacity level for the ability to execute a motor activity with spatial and temporal accuracy is directly related to the spatial and temporal distance between the dropping and catching hands and eccentricity of any spatial offset between them as well as the temporal duration between the dropping and catching actions. Critically, each challenge may have one or more partial success cases. In the example above, shooting to a location previously occupied by a target after the target disappeared, or missing an existing target may count as partial successes, since this prompted the subject to action, albeit that the action was incorrect or inaccurate. In some embodiments herein, an analysis of partial successes by the algorithms includes a determination during a play session which ability was sufficient to enable subject success, and which abilities were insufficient, leading to subject failure. For example, in the case of a subject shooting at a target that has disappeared (i.e. a late hit) the subject is considered to have succeeded at the challenge of identifying the object, but failed at responding in time. The success and failure of these individual abilities are logged within the subject's profile.

In some embodiments herein, the specific ability levels used to generate a challenge are determined by the subject's recent successes and failures within the relevant abilities. To determine the ability levels used within a challenge, the methods, tools and systems herein create a measure of the subject's current cognitive ability, i.e. thresholds, within a particular ability and context, and provide challenges near those thresholds. For example, a measure of a subject's cognitive ability can include measurement of a spatial crowding threshold, a temporal crowding threshold, a UFOV, an imprecision threshold or any combination thereof. By accurately providing challenges near and slightly above a subject's ability level, therapeutic benefit and challenge is provided. In some embodiments, challenges below the subject's ability will provide some rest from the intensity of the exercise, but likely will not carry therapeutic benefit provided by challenges near the thresholds.

In some embodiments herein, the system includes a generation algorithm that collects and utilizes input of user responses and generates data that is then utilized by the system to produce one or more outputs, such as a new prompt to the subject (user) for an action (i.e., prompts such as an object, event, or target that generates an action such as behavior or response from the subject). In some embodiments, the generation algorithm takes the history of the user's responses to specific prompts as inputs and produces a set of spatial/temporal/physical data that is used to generate further prompts to action. The user's responses to those subsequent prompts are included in the calculation of future prompts to action, and this cycle of input, calculation and subsequent prompt(s) may be repeated. The data produced by the algorithm is used to make prompts to action more or less difficult as it pertains to a particular physical motion or cognitive ability that is being assessed and treated.

In one example, consider a subject (user) who is limited in the distance in which they can reach forward. The generation algorithm will produce values representing distances that the user may be able to reach forward, given the state of the subject's disability. Those values will then be used by the system to place virtual objects at the given distance away from the user, who will then be prompted to reach forward to touch the object. Subsequent iterations of the generation algorithm will consider the user's performance in the task of reaching forward (e.g. how far the user ultimately reached, the path which the user's hand took to reach, the speed and acceleration of the hand along the path, etc.) in generating new values.

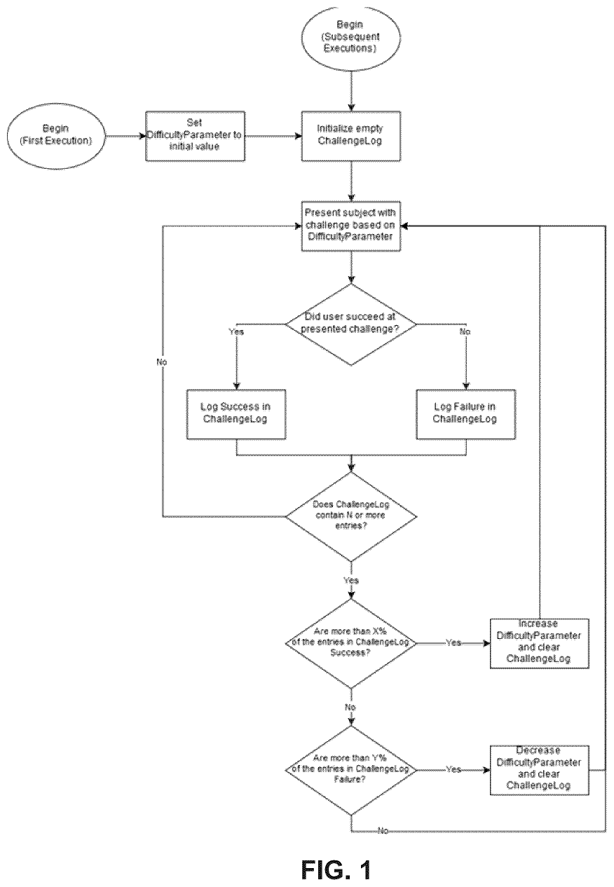

In some embodiments of the generation algorithm, the system maintains a value that represents a prediction of the user's maximum ability to perform the desired action, as well as a historical log of the player's binary success and failure at performing the action given generated values. Each iteration of the algorithm creates a sample set of a given size of this history and generates an overall success rate for actions with similar values. If the success rate is above or below a specified range, the algorithm adjusts the predicted maximum ability value to compensate for a challenge that is too easy or too difficult, respectively. In some embodiments, if the success rate is above 50%, 55%, 60%, 65%, 70%, 75%, 80%, 85%, 90%, 95%, or greater than 95%, the system can adjust to increase the difficulty level. Similarly, if the success rate is below 60%, 55%, 50%, 45%, 40%, 35%, 30%, 25% or lower than 25%, the system can reduce the level of difficulty. In some embodiments, if the success rate is above 70%, the system increases the level of difficulty and if the success rate is below 30%, the system reduces the level of difficulty. Finally, the algorithm returns the predicted maximum ability value to be used in generating the prompt to action. An exemplary embodiment of a generation algorithm is shown in FIG. 1.

In some embodiments, the system maintains a historical log of the player's binary success and failure at performing the action given generated values. Each iteration of the algorithm creates a sample set of a given size of this history. This set is then treated as the sample set for a process of Bayesian inference which seeks to predict the user's current maximum ability. The prior probability for the Bayesian inference may be based on previous predictions of the user's maximum ability or the maximum abilities of other subjects with demographics similar to the user. Once the maximum ability value is predicted, it is used to generate the prompt to action. For example, each challenge is considered a sample of the subject's actual ability, which is treated as a random variable, and the subject's success or failure along with the challenge's ability level contributes to a probability distribution describing what the subject's ability may be. In one embodiment, a method of predicting the subject's ability relies on machine learning that may be done online by a game client, or offline by a remote data storage and processing service. Machine learning is employed to create an agent capable of generating challenges on a per-subject basis using the subject's success and failure rates as training features.

The machine learning algorithm may be a supervised machine learning algorithm. A supervised machine learning algorithm may be trained using a set of labeled training examples, i.e., a set of inputs with known outputs. The training process may include providing the inputs to the machine learning algorithm to generate predicted outputs, comparing the predicted outputs to the known outputs, and updating the algorithm's parameters to account for the difference between the predicted outputs and the known outputs. For example, the machine learning algorithm may be trained on a large sample of success rates at various difficulty levels. The success rates may be previous success rates of the user or previous success rates of similar subjects, e.g., subjects with the same neurocognitive disorder. The labels for this training data may be known maximum ability levels of the user or similar subjects based on clinical or other testing.
Neural networks are one class of supervised machine learning algorithm. Neural networks may include feedforward neural networks (such as convolutional neural networks) and recurrent neural networks (RNNs). A neural network may be trained to predict or classify a user's maximum ability level by comparing predictions made by its underlying machine learning model to a ground truth. An error function may calculate a discrepancy between the predicted value and the ground truth, and this error may be iteratively backpropagated through the neural network over multiple cycles, or epochs, in order to change a set of weights that influence the value of the predicted output. Training may cease when the predicted value meets a convergence condition, such as obtaining a small magnitude of calculated error. Multiple layers of neural networks may be employed, creating a deep neural network. Using a deep neural network may increase the predictive power of a neural network algorithm.
Additional machine learning algorithms and statistical models may be used in order to obtain insights from the types of input data disclosed herein. Additional machine learning methods that may be used are logistic regressions, classification and regression tree algorithms, support vector machines (SVMs), naive Bayes, K-nearest neighbors, and random forest algorithms. These algorithms may be used for many different tasks, including data classification, clustering, density estimation, or dimensionality reduction. Machine learning algorithms may be used for active learning, supervised learning, unsupervised learning, or semi-supervised learning tasks. In this disclosure, various statistical, machine learning, or deep learning algorithms may be used to predict a user's maximum ability level.

In some embodiments, the system as described herein also analyzes the user's physical motion as they attempt to complete actions prompted by the system. The analysis generates a value that describes how difficult the user found the action to perform. This value replaces the binary success/failure value in the above algorithm, with the Bayesian inference portion seeking to predict a value which will provide to the user a given level of difficulty.

One measurement that may be employed with the system herein to assess a subject's ability is based on data gathered previously from others with similar demographics, as well as analysis of the current subject's past performance. In one implementation of this analysis, a player's ability level is set at a particular value and challenges are created based on that assumption. After a predetermined number of challenges, the player's successes and failures within that ability are tabulated, and an increase or decrease is made to that assumed value depending on whether the subject's success rate was above or below certain desired thresholds. For instance, a subject receives slightly more spatially-eccentric targets to shoot at after correctly hitting 8 of the last 10 targets shown.

In the methods, and systems provided herein include tools such as data structures and code that may be stored on a computer-readable storage medium, including any device or medium that can store code and/or data for use by a computer system. The computer-readable storage medium includes, but is not limited to, volatile memory, non-volatile memory, magnetic and optical storage devices such as disk drives, magnetic tape, CDs (compact discs), DVDs (digital versatile discs or digital video discs), or other media capable of storing computer-readable media now known or later developed. The data structures, data and code can be stored in such computer-readable storage medium. When a computer system reads and executes the code and/or data stored on the computer-readable storage medium, the computer system performs the methods and processes embodied as data structures and code and stored within the computer-readable storage medium. Other components for use with the methods, tools and systems herein may include hardware modules. For example, the hardware modules can include, but are not limited to, application-specific integrated circuit (ASIC) chips, field-programmable gate arrays (FPGAs), and other programmable-logic devices now known or later developed. When the hardware modules are activated, the hardware modules perform the methods and processes included within the hardware modules.

In some embodiments, the computer-based processing, analyses or responses include one or more algorithms. In some embodiments, some or all of the calculations carried out by the algorithms determine placement and involve determining the size, in degrees of visual angle (DVA) of targets, flankers and other relevant objects in the environment. Determining DVA involves having values for the size of the target object and the distance from which it is being viewed.

The methods, tools and systems herein may include additional hardware to deliver the VR environment. In some embodiments, the VR environment is presented to the subject through the use of goggles, a helmet, glasses or other visual-ware that provides for a three-dimensional representation of images. In some embodiments, a device for projecting sound, including placing sound cues in a three-dimensional context and/or in response to a subject's movements is included. The hardware for use herein may include one or more cameras to monitor, record or capture a subject's movements. In some embodiments, the hardware includes sensors that track physical movements of one or more of a subject's body parts. In some embodiments, the hardware includes sensors that monitor, record or capture a one or more of a subject's physiological responses. In some embodiments, the methods, tools and systems herein include a computer-based processing or analysis of a subject's responses, including one or more physical, physiological, emotional or cognitive responses. In some embodiments, the methods, tools and systems herein include a computer-based response to a subject's response. In some embodiments, the computer-based response includes one or more alterations to the VR environment.

In some embodiments of the methods, tools and systems provided herein, the VR employed includes a constant viewing distance such as by a fixed screen in the headset or other employed viewing hardware affords improved accuracy in DVA calculation. In such constant viewing distance embodiments, the DVA calculation accuracy is improved over stimuli presented in two-dimensional format such as on a computer monitor or tv screen because variables including view position, height, head and body movement result in coarse DVA calculations with error introduction when performed in these two-dimensional systems.

In some embodiments, the system herein includes variables that specify values of spatial and temporal information and the combination of spatial and temporal information. For example, variables may include one or more spatial measurements based on the position of one or more components of a user's body and one or more spatial measurements of a user's movement in response to a prompt. For example, variables may be calculated in spatial terms such as how high or low, or how far to the right or left of the user's midline can the user reach with an arm or hand at the time when the ability estimate is calculated by the system.

In an exemplary embodiment, the system includes data in the form of a timeseries of positional information of the head-mounted device (HMD) and input devices (such as handheld controllers, gloves or other hardware) generated by the user's movements and actions. In some embodiments, positional data is captured at a high rate (e.g. 20-50 Hz) by cameras mounted in the HMD, or other sensors, that allow 360-degree spatial measurement over time.

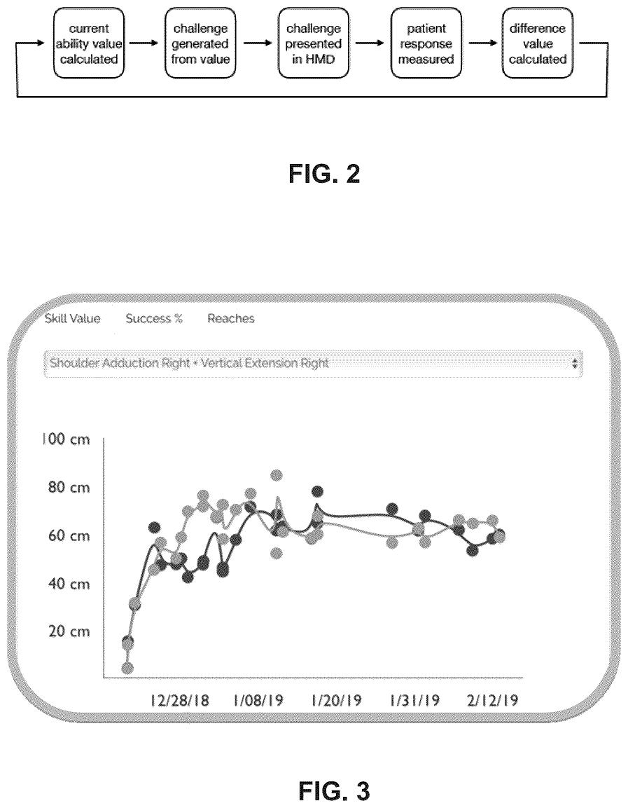

In one example, the general process for all such calculations is depicted in FIG. 2. The level of challenge that is necessary to change those values consistent with improved ability is calculated as follows. First, an event is generated that requires a mental or physical response from the user that is relevant to the goals of the specific treatment activity. The generation algorithm takes as inputs the current values of the functionality estimate and produces an event that requires a response within the range of the lowest to highest ranges of the current ability (box 2). This event, which comprises a combination of visual, auditory, and possibly tactile, information with a specific duration, is then presented to the sensory systems of the user via the VR system hardware elements. The user's cognitive and mental processes in reaction to that event with respect the goals of the activity will generate some kind of behavioral output (including, but not limited to, physical movements of the head and/or other body parts, physical movements of the eyes, changes in respiration rate or other physiological responses such as pupil dilation/contraction, galvanic skin response, etc.) that can be detected by the VR system hardware (box 3). The algorithms then take as inputs the values generated by the response(s) and compares them to the values of the existing estimate (see box 1) in order to generate as outputs a difference value (box 4). This is then fed back into the existing ability estimate to calculate an updated value (that may or may not differ from the existing one, depending on the progress of the user).

Positional information from the user (i.e., inputs to the system) may include for example transceivers, cameras, and/or sensors. In one embodiment, hand controllers contain ultrasonic transceivers which allow them to determine their position relative to the headset by measuring the time delay of response from the headset. In another embodiment, the headset contains a set of infrared cameras arranged to capture images from a certain field of view around the player. The hand controllers, in turn, have infrared LEDs embedded within them that can be tracked by the cameras. The images from the cameras are analyzed to determine how far away the LEDs are from the camera, and in which direction. This, in concert with inertial measurement units (IMUs) on the hand controllers, determines the relative position and orientation of the hands.

In one embodiment, the player attaches IMUs to specific points on their bodies. The IMUs wirelessly connect to the headset and transmit orientation data on a moment-to-moment basis. During initial setup, the patient inputs specific body dimensions (such as forearm length and shoulder span) that describe the upper limb. The system then estimates the position of the patient's hands using an inverse kinematics solver that consumes the orientation and body dimension data. In another embodiment, the absolute head position is determined by an array of depth sensing cameras embedded in the headset. These depth sensing cameras project infrared or near-infrared lasers out from the headset and capture that light as it reflects off nearby surfaces. Using those data, the headset computes a depth map detailing its current distance from every nearby surface, and uses that depth map to determine its absolute position from moment to moment. Hand position is then determined by one of the above methods.

Positional information for a subject's trunk may be measured directly or indirectly. For example, in some embodiments the system tracks head and hands only, and trunk position is estimated based on the dimensions of the subject's body. Such dimensions may be inputted by the subject or an observing clinician or alternatively, the system can calculate the dimensions based on data obtained from the subject's interaction with the system, such as through sensors, cameras interpolation of positioning of one or more body parts to calculate the dimensions of one or more body parts. The estimation comes from an inverse kinematic model with typical human joint constraints. In some embodiments, the system includes additional tracking points in addition to head and hands. For example, a subject may be provided a belt worn on the chest with a tracking module on it. The system will poll the tracking module's orientation and factor that into a calculation of trunk position.

Variables can include calculations in temporal terms, such as how quickly after the appearance of an object in the display or an instruction to respond to such an object, can initiate the action, how quickly can the user execute the action, and what is the latency required before the user can initiate a new action. Such measurements can include measurements by the cameras and other sensors that comprise the VR hardware.

In some embodiments, input data from a subject combines spatial and temporal information. For example, the system may measure the speed at which the user can initiate and execute a movement to a specific location in space, how stable is the movement that is required (i.e. how much variation from a direct spatial path is measured (how “wobbly” is the action) as well as how much variation from a consistent speed in measured (how “smooth” is the action), how accurate is the movement (i.e. how far from the intended target location does the action terminate and does it need successive adjustments over time).)

The system may utilize positional and/or temporal information as input pertaining to the subject and for output of images, sound and challenges to the subject. In some embodiments, the system determines on a moment to moment basis, the position and orientation of the subject's head. Using the position and orientation of the head, the system positions a “virtual camera” to render the virtual environment (e.g., visual images) from the subject's perspective. The virtual camera renders the environment and provides render data to the headset in such a way as it enables binocular vision for the subject. The position and orientation of the virtual camera is also used to determine audio levels in a way that simulates spatialized sound.

In some embodiments, the system determines on a moment to moment basis, the position and orientation of the subject's hands. Using the position and orientation of the hands, the system places virtual hand models in the virtual environment so that they match the real world position and orientation of the subject's hands. These virtual hands appear to the subject as if they were the subject's real hands; in some instances they may be colored or shaped differently from normal human hands. The system tracks the position and orientation of the patient's hands on a moment-to-moment basis and logs this data to a file which may be used for analysis. In one example of such embodiments, the timeseries data of the patient's hand and head movements is cross-referenced with other events that occur within the system, such as the appearance of certain objects, the beginning and end of specific challenges, and interactions between objects such as collisions.

In some embodiments, the timeseries data of the patient's hand and head movements are taken as inputs by signal processing and/or classifier algorithms that produce metrics related to aspects of the subject's treatment progress and recovery. The metrics produced by the algorithms are then consumed by the system to determine optimal difficulty settings for certain challenges. The metrics produced by the algorithms may be made available to the subject as a motivational tool. The metrics produced by the algorithms may be made available to clinicians as a diagnostic tool.

In some embodiments, the positional information collected by the system related to hand, head and/or trunk is utilized to present challenges to the subject. In one example, the system presents challenges to the subject that must be completed by moving a hand and/or arm in a specific way. The system consumes the position and orientation data of the hand (or hands) and head to determine if the subject successfully completes the challenge. In one instance of these challenges, the subject is tasked with supinating (turning outward) their forearm. The system considers the challenge complete if the relevant hand controller is rotated so the palm is facing upward. In another exemplary instance of these challenges, the patient is tasked with touching a specific virtual object. The system considers the challenge complete if the relevant hand controller is moved to the same position as the virtual object.

In some embodiments, the spatial and/or temporal information collected specifies values of spatial and/or temporal crowding threshold estimates of the subject by creating values for the spatial and/or temporal separation of objects or events occurring in the display that are necessary to ensure the ability of the subject to respond appropriately during the specific play session. Variables compute the temporal separation required between events necessary to ensure that the subject can accurately respond at some target rate (e.g. 80%) to each one. When separation is too low and the response rate is below the threshold, temporal separation is increased. When temporal separation is too high and the response rate exceeds the threshold, temporal separation is decreased (such that the challenge level is higher). Similarly, variables compute the spatial separation required between objects necessary to ensure that the subject accurately respond at some target rate (e.g. 80%) to each one. When spatial separation is too low and the response rate is below the threshold, the spatial separation is increased. When spatial separation is too high and the response rate exceeds the threshold, spatial separation is decreased (such that the challenge level is higher).

### EXEMPLARY EMBODIMENTS

The following are exemplary embodiments of the methods, tools and systems provided herein and are not intended to be limiting on the implementation of such methods, tools and systems.

### Example 1

In one example, a treatment is constructed and delivered as an FP-POV three-dimensional VR in a format of a fantasy sports training environment. Such environment includes one or more “mini-games”, each designed to deliver at least one element of an active compound. In one such exemplary mini-game, the subject finds him/herself in an environment resembling a “pod” or compartment of a spaceship. There are four “storage ports” near the subject. One is immediately above the subject in the ceiling, one is in the floor just in front of the subject and the other two are in the walls of the pod immediately to the subject's right and left. The subject is facing a wall, within which are several “launchers” the fire balls that change color with subject's progress (and occasionally are replaced by unexpected objects like chickens). As each ball is launched, it is tracked by a floating/flying indicator showing into which port the ball must be thrown after it has been caught. The subject must then catch the ball and toss it into the indicated port (i.e. providing an objective measure of his/her ability to detect, decode and act upon the information carried by the indicator). Various play characteristics are manipulated by the treatment algorithms in response to subject actions that allow highly sensitive, dynamically adapted, targeted neurocognitive stimulation to be delivered.

For example, in one embodiment, the indicator is an arrow which is flanked by two very similar lines that can cause spatial crowding of the indicator. When crowded, the indicator will be detectable but enough information will be lost that the resulting mental representation cannot be decoded in sufficient detail to allow the subject accurately determine which is the target port. Therefore, performance accuracy will drop on crowded trials. Spatial crowding thresholds can be approached or exceeded in several ways, including manipulating the spatial separation of the indicator arrow and the flanking lines, changing the duration of the indicator's and flankers' presence on the screen, altering the eccentricity in the UFOV of the indicator from the subject's attentional focus (i.e. the approaching ball), among others.

### Example 2

In one example of the methods, tools and systems provided herein, the subject acts as the apprentice to a powerful wizard. As the apprentice, the subject must assemble magical components and perform hand gestures to help the wizard cast spells. The specific tasks the subject needs to execute include exercises designed to help reduce spatial imprecision. One such task may require the subject to pick up a vessel, fill it with magic potions, and pour them into a cauldron. To begin this activity, the subject must reach out with the VR hand controller and squeeze a trigger to indicate he/she intends to grab an object (ideally, the vessel). At this point, the algorithms calculate the distance between the subject's virtual hand and the object he/she is attempting to grab. If the distance is above the subject's grabbing threshold (regardless of whether or not the subject is actually touching the object), the subject will pick up the object and be able to move it around as such. If the distance is below the subject's grabbing threshold (i.e. their hand is too far away), the individual will not pick up the object. As the subject successfully grabs objects, the calculations performed by the algorithms lower the threshold, and as the subject fails to grab objects, the algorithms raise the threshold. Such adjustments result in the reduction of spatial and temporal imprecision in the subject (e.g. a stroke patient) allowing for reconstruction or retraining of lost functioning or enhanced performance in the subject (e.g. an athlete or a person with a neurodevelopmental disorder that impairs the motor function).

A similar protocol can be applied to the act of pouring the potions into the cauldron. When the subject tips the vessel to pour out its contents, the system creates a measurement of the distance between the vessel and the cauldron and determine if it meets a separate threshold criterion. If the distance is above the threshold, the subject receives credit for successfully pouring the liquid (which may be visually represented by the liquid coming out with some lateral velocity to land in the cauldron). If the distance is below the threshold, the subject will not receive credit for pouring the liquid, and the liquid will end up on the floor.

### Example 3

In one example, mini-games are used to enhance temporal resolution (i.e. reduce temporal crowding). Temporal crowding is a component of, among other things, the “attentional blink” phenomenon. This describes the duration of the gap between two temporally-spaced targets appearing in a similar spatial location at which the second of the two items is detected by the sensory system but cannot be processed in sufficient detail to distinguish it from the first target, processing of which has yet to be completed.

In some embodiments for temporal resolution, instructions in the game may indicate, for example, that objects, like a colored ball that is not red, should only be treated as a target when it is preceded by the launching of a red ball. Temporal crowding thresholds can be approached or exceeded in several ways, including manipulating the temporal separation of the two balls, changing the duration of one or both of the balls' presence on the screen, altering the eccentricity in the UFOV of the balls from the subject's attentional focus (e.g., some secondary activity such as monitoring a central location for a specific signal).

In some embodiments for temporal resolution, include a mini-game that require the subject to pick up an object, hold it at an elevated level, and drop it, and catch it with the other hand. This exercise challenges the subject's sense of temporal resolution as the subject must accurately and precisely judge the time it will take for the object to fall into one hand so they can catch it. It also challenges spatial resolution as the dropping and catching hand must be aligned in space or the ball will drop beyond the grasp of the catching hand. The subject must place the hand in the appropriate place and, in the case of the use of a controller, squeeze a trigger to indicate the attempt to grab the falling object, or simply close the catching hand if some other tracking system is being used. At the time the subject squeezes the trigger or drops the ball, the system calculates the distance between the virtual hand and the falling object. If that error term in the calculation of that distance is above a certain value, the subject will not catch the object. However, if the error term in the calculation of that distance is below the value (i.e. the object is closer to the hand than the threshold) the subject will successfully catch the object (represented by the object snapping to an appropriate position in the virtual hand).

### Example 4

In one example of a mini-game includes increasing the UFOV of the subject. The UFOV variables are calculated as any viewable space on the VR screen (or viewing medium) and the overall field is defined as a set of concentric circles (not visible to the participating subject). Each circle subtends a size of a specified number of DVA (i.e. 1 or 2 degrees), can be further subdivided into much smaller concentric circles for more finely tuned stimulation. By distributing potential targets with equal probability across the total field of view, the systems, methods and tools herein induce an adaptive response of the subject, which is to focus on the center of the display. The potential targets can be presented within any concentric circles, requiring a response from the subject. If the desired response is produced, this provides an objective measure of the object's detection, which is then fed back into the algorithms to determine the optimal placement, and duration, of subsequent targets.

In one such mini-game, the subject participates in a variant of the Olympic Biathlon where the individual alternates climbing vertical distances and shooting targets with a rifle within a time limit. Throughout the climbing sections, the subject must grab and pull on hand-holds which appear at specific locations in the individual's vision. After climbing for a certain distance, the subject must then grab a rifle, and begin shooting at targets that appear (and eventually disappear) within the visual field in a similarly computed manner as the hand-holds. Spatial eccentricity detection thresholds (i.e. UFOV limits) can be approached or exceeded in several ways, including increasing the eccentricity in the UFOV of the hand-holds and targets from the center of the subject's vision, manipulating the temporal duration of the hand-holds and targets on the screen, and presenting multiple targets at once.

### Example 5

Parameter values for the methods, tools and systems provided herein include initial values for which are then changed in a personalized and continually adaptive fashion for the subject being treated during the session. Values are determined through small pilot testing studies with the specific population to which any given instantiation of the treatment is targeted. Data collection combines statistics such as acceptance and adoption (e.g. is the “game” played, how often, for how long . . . ) and preliminary efficacy measures using research- and clinically-validated measurement instruments.

When supra-threshold targets are perceived, represented and responded to (as determined by the rules of the “game”), the difficulty is escalated by increasing difficulty in using the thresholding measures such as one or more of those described herein. When targets are successfully perceived, represented and responded to at increased spatiotemporal complexity, the values of relevant parameters are taken as inputs to the treatment algorithms and the difficulty is gradually increased (or decreased) in a constant ongoing adaptive fashion.

### Example 6

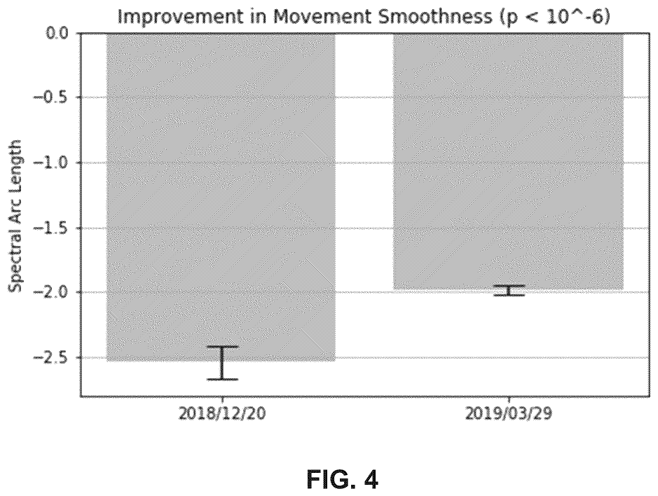

A VR system as described herein was employed for neurocognitive therapy with a 64-year old male, 6 years post hemorrhagic brainstem stroke with hemiplegia of the right side. A commercial off-the-shelf (COTS) VR system set up at the subject's home in a rural community. The system comprised an Oculus “Rift” headset with 6 degrees of freedom “Touch” hand controllers. The system was powered by an unmodified Dell VR-compatible laptop upon which a version of the Cognivive VR treatment software was installed. The headset was connected to the computer with a combined HDMI and USB cable and the battery-powered hand controllers were connected wirelessly to the same hardware. Tracking of the movements of the headset and hand controllers was achieved via the sensor towers provided as part of the Oculus system and connected via USB cables. A short demonstration of the hardware and software was provided and then the subject was left to use the system thereafter unsupervised. Prior to implementation of the VR system, the subject was performing very few rehabilitation exercises with any regularity. After the VR system was provided, the subject, based on his own motivation and self-reported enjoyment of the treatment games, performed by self-administered VR system treatment over a 3-month period. He reported limiting himself to 30 minute sessions per day because, with very limited sensation in his right arm and hand, he was unsure if he could induce some kind of further injury from a sudden transition to intensive use of the upper extremity. Data from all of his activity with the installed system was collected and stored on the hard drive of the computer and, despite challenging internet connectivity conditions due to the rural setting, was transmitted to a remote server whenever connectivity allowed and was subsequently analyzed. In the first two months of having the system installed in his home the subject had accrued 59 interaction sessions with the system. Within those sessions he had completed 50 different activities that included 5,197 treatment-related actions. All of this activity represented 10 hours, 41 minutes and 56 seconds of engagement with the treatment system. The subject reported “loving” the treatment games has was playing. During this time, numerous measures of upper extremity range of motion against gravity increased considerably. Vertical extension of the right (affected) hand changed from about 20 cm, measured in the earliest interactions with the system, to about 80 cm at their peak with stability at about 60 cm at the end of the 2 month period, presumably due to increasing challenge or difficulty of the required activities over time. Right (affected) shoulder adduction, which is rotation of the joint allowing the affected arm to be moved across the midline of the body followed a similar track and produced similar range of motion values (shown in FIG. 3). As is characteristic in post-stroke neuromotor control, the subject's intentional task-related affected arm movements that were the target of treatment were very unstable and “jerky”. One measurement of instability, or jerkiness around the center of the intended path, is called the spectral arc length. Three months after initial use of the system, the subject's spectral arc length for a measured treatment activity had reduced from −2.5 to just under −2.0 indicating a highly statistically significant increase (with probably of this being a chance outcome of p<10⁻⁶) in movement smoothness (shown in FIG. 4).

### Example 7

The VR system as described herein was deployed in several in-office usability and design consultation (playtesting) sessions with 5 subjects with range of abilities but all with L or R hemiplegia, limited control of at least the affected side upper extremity, and minimal sensory input from that upper extremity. They ranged in age from 39 to 63 years of age and were between 7 months and 8 years of post-stroke recovery. All subjects were able to use the treatment games that were updated versions the one used by the patient described in Example 6 and were deployed using identical hardware. Part of the purpose of the sessions was to optimize the motivational and usability aspect of the treatment games by including a sample of representative “end users” (i.e. stroke patients) in that process to provide feedback about how much the enjoyed, understood, were motivated by, could navigate and were able to respond appropriately (among other things) to the treatment game prototypes. A standard set of video game “playtesting” questions was administered before, during and after use by each patient of the treatment games by the leader of the design team. Information was also gathered about the limitations of the COTS hand controllers, particularly with respect to whether the subjects could grip, hold, manipulate and not drop them, so that design changes in software and requirements for different and/or novel types of hardware, such as trackable VR gloves or other sensors attached to body parts could be used and/or designed. All subjects provided very positive assessments of the concept and design of the VR treatment games, including comments such as “this is addictive”, “I'd pay a lot of money to have this”, and “this is better than Candy Crush”. Some subjects, without prompting, described the phenomena of having the hand they are controlling in space “make more sense” within the VR system than in real life, thereby indicating treatment was likely opening new sensorimotor neuro-motor pathways. An expert neurorehab clinician observed the subjects' sessions and reported “amazement” at amount of engagement and motivation shown by subjects.

1. A method for enabling a subject to experience neurocognitive therapy through a virtual-reality (VR) interface to improve spatial and/or temporal information-processing capabilities of the subject, the method comprising:
providing a system comprising a neurocognitive therapy environment having a VR interface, wherein the neurocognitive therapy environment operates by:
displaying spatial and/or temporal information in the form of one or more target items or events to the subject through the VR interface;
measuring a response of the subject to the spatial and/or temporal information; and
using a measurement of the response to adaptively control a feature of the neurocognitive therapy environment selected from the group consisting of a visual angle distribution, a spatial distribution, a temporal presentation rate, and any combination thereof.

2. The method of claim 1, wherein the subject's spatial and/or temporal information-processing capabilities are enhanced by treatment with the neurocognitive therapy environment.

3. The method of claim 1, wherein measuring the response of the subject to the spatial information includes determining a visual angle crowding threshold for the subject based on how far apart in visual angle two target items can be while still perceived as distinct unitary items by the subject.

4. The method of claim 1, wherein measuring the response of the subject to the temporal information includes determining a temporal crowding threshold for the subject based on how short a duration between appearances of two target items can be while still being perceived as distinct unitary items by the subject.

5. The method of claim 1, wherein measuring the response of the subject to the spatial information by the subject includes determining a useful field of view (UFOV) threshold for the subject, which measures a distribution in space, viewed at a specified distance, over which the subject's attention can be spread.

6. The method of claim 1, wherein adaptively controlling the feature of the neurocognitive therapy environment comprises:
presenting a first presented set of one or more target items or events so that spatially distributed and temporally proximate target items or events are initially presented within a determined visual angle crowding, temporal crowding and/or UFOV thresholds of the subject; and then
adaptively presenting a second set of target items or events close to the determined visual angle crowding, temporal crowding and/or UFOV thresholds of the subject to stimulate enhancement of visual angle, temporal and UFOV discrimination abilities of the subject.

7. The method of claim 6, wherein adaptively presenting the target items or events close to the visual angle crowding thresholds of the subject comprises:
increasing the visual angle between target items or events in the second set when the subject successfully responds to less than a threshold percentage of the first presented set of target items or events; and
decreasing the visual angle between target items or events in the second set when the subject successfully responds to more than the threshold percentage of the first presented set of target items or events.

8. The method of claim 6, wherein adaptively presenting the target items or events close to the temporal crowding thresholds of the subject comprises:
decreasing a duration between appearances of target items or events in the second set when the subject successfully responds to a more than threshold percentage of the first presented set of target items or events; and
increasing a duration between appearances of target items or events in the second set when the subject successfully responds to less than the threshold percentage of the first presented set of target items or events.

9. The method of claim 6, wherein adaptively presenting the target items or events close to the UFOV thresholds of the subject comprises:
increasing a distribution in space between target items or events in the second set when the subject successfully responds to less than a threshold percentage of the first presented set of target items or events; and
decreasing a distribution in space between target items or events in the second set when the subject successfully responds to more than the threshold percentage of the first presented set of target items or events.

10. The method of claim 1, wherein the system comprises a VR headset and facilitates a 360-degree presentation of target items or events.

11. The method of claim 1, wherein the system receives three-dimensional inputs from one or more input devices, which track a three-dimensional position and/or orientation of one or more body parts of the subject.

12. The method of claim 1, wherein the system receives three-dimensional inputs from a pointing device that facilitates three-dimensional tracking, which is manipulated by the subject.

13. The method of claim 1, wherein measuring the response of the subject comprises processing one or more three-dimensional inputs received by the system to reconstruct three-dimensional positions, motions and/or actions associated with the subject.

14. The method of claim 1, wherein the spatial information is the distance between at least 2 target items or events displayed by the VR interface.

15. The method of claim 14, wherein the VR interface displays 2, 3, 4, 5, 6, 7, 8, 9, 10 or more than 10 target items or events concurrently.

16. The method of claim 14, wherein the spatial information is the distance in 3-dimensional space between 2 target items or events.

17. The method of claim 1, wherein the temporal information is the time between display of a first target items or event and display at least one second target item or event by the VR interface.

18. The method of claim 17, wherein the VR interface displays 2, 3, 4, 5, 6, 7, 8, 9, 10 or more than 10 target items or events sequentially.

19. The method of claim 17, wherein the temporal period between the sequential display 2 target items or events.

20. The method of claim 1, wherein the neurocognitive therapy environment measures a series of responses of the subject to the spatial and/or temporal information.

21-65. (canceled)
